(19) United States
(12) Patent Application Publication
(10) Pub. No.: US 2026/0014122 A1
(43) Pub. Date: Jan. 15, 2026

(54) DRUG FOR LOWERING BLOOD LDL CHOLESTEROL

(71) Applicant: KOWA COMPANY, LTD., Nagoya-shi (JP)

(72) Inventor: Ryohei TANIGAWA, Chuo-ku (JP)

(73) Assignee: KOWA COMPANY, LTD., Nagoya-shi (JP)

(21) Appl. No.: 18/880,136

(22) PCT Filed: Jul. 14, 2023
(86) PCT No.: PCT/JP2023/025980

§ 371 Date: Dec. 30, 2024

(30) Foreign Application Priority Data
Jul. 15, 2022 (JP) 2022-113930

(51) Int. Cl. A61K 31/423; A61P 3/06

(52) U.S. Cl. CPC A61K 31/423; A61P 3/06

(57) ABSTRACT

A novel preventive and/or therapeutic agent useful in the prevention and/or treatment of a disease on which a therapeutic effect can be expected by a reduction in blood LDL-C. The present invention relates to a medicine for preventing and/or treating a disease on which a therapeutic effect can be expected by a reduction in blood LDL-C, the medicine including a therapeutically effective dose of (R)-2-[3-[[N-(benzoxazol-2-yl)-N-3-(4-methoxyphenoxy)propyl]aminomethyl]phenoxy]butyric acid or a salt thereof, or a solvate thereof.

### FIELD OF THE INVENTION

The present invention relates to an agent for reducing blood LDL cholesterol.

### BACKGROUND OF THE INVENTION

Many epidemiological investigations have shown that the onset rate and mortality rate of a coronary artery disease increase along with an increase in blood LDL cholesterol (LDL-C). In recent years, it has been successively reported that an active reduction in blood LDL-C suppresses the onset of a cardiovascular event in a patient having a risk of the onset of the coronary artery disease. Accordingly, a therapy for reducing the blood LDL-C has been regarded as being clinically important. In view of such background, in medical guidelines in various countries, a target for the management of the blood LDL-C has been set in accordance with the risk of the onset of the coronary artery disease (Non-Patent Documents 1 to 3).

Improvements in lifestyle habits including a diet therapy and an exercise therapy are fundamental to the management of the blood LDL-C. However, a drug therapy is required for a patient in whom it is difficult to manage the blood LDL-C only through the improvements in lifestyle habits. In the drug therapy, an HMG-CoA reductase inhibitor (hereinafter referred to as “statin”) has been recommended as a first-choice drug. However, in many patients, target values for blood LDL-C management may not be achieved only with statin, and hence addition of a therapeutic drug except statin, such as ezetimibe or a PCSK9 inhibitor, is required for these patients (Non-Patent Documents 1 to 3). In addition, there exist a certain number of patients in whom administration of statin is not to be continued owing to side effects, such as a muscle-related side effect and liver damage, and hence a therapeutic drug except the statin, such as ezetimibe or the PCSK9 inhibitor, is also required for these patients (Non-Patent Documents 4 and 5). However, the alternatives of drug therapies intended for reduction in blood LDL-C are limited, and hence a novel therapeutic drug may be useful in sufficiently managing the blood LDL-C.

Meanwhile, Patent Document 1 discloses that a compound of the following formula (1) or a salt thereof, or a solvate thereof has a selective PPARα-activating effect, and is hence useful as a preventive and/or therapeutic agent for diabetes, a diabetic complication (e.g., diabetic nephropathy), inflammation, a heart disease, or the like that is not accompanied by a weight gain or obesity in a mammal including a human:

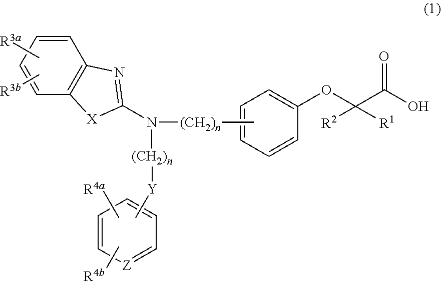

where R¹ and R² may be identical to or different from each other, and each represents a hydrogen atom, a methyl group, or an ethyl group, R^{3a}, R^{3b}, R^{4a}, and R^{4b} may be identical to or different from each other, and each represents a hydrogen atom, a halogen atom, a nitro group, a hydroxy group, a C₁₋₄ alkyl group, a trifluoromethyl group, a C₁₋₄ alkoxy group, a C₁₋₄ alkylcarbonyloxy group, a di-C₁₋₄ alkylamino group, a C₁₋₄ alkylsulfonyloxy group, a C₁₋₄ alkylsulfonyl group, a C₁₋₄ alkylsulfinyl group, or a C₁₋₄ alkylthio group, or R^{3a} and R^{3b}, or R^{4a} and R^{4b} are bonded to each other to represent an alkylenedioxy group, X represents an oxygen atom, a sulfur atom, or N—R⁵ (R⁵ represents a hydrogen atom, a C₁₋₄ alkyl group, a C₁₋₄ alkylsulfonyl group, or a C₁₋₄ alkyloxycarbonyl group), Y represents an oxygen atom, a S(O)ₗ group (“l” represents a number of from 0 to 2), a carbonyl group, a carbonylamino group, an aminocarbonyl group, a sulfonylamino group, an aminosulfonyl group, or an NH group, Z represents CH or N, “n” represents a number of from 1 to 6, and “m” represents a number of from 2 to 6.

However, there is no description or suggestion of what effect such compound has on blood LDL-C.

### PRIOR ART DOCUMENTS

### Patent Document

[Patent Document 1] WO 2005/023777 A1

### Non-Patent Documents

[Non-Patent Document 1] Japan Atherosclerosis Society (edition): Japan Atherosclerosis Society (JAS) Guidelines for Prevention of Atherosclerotic Cardiovascular Diseases 2017, Japan Atherosclerosis Society, 2017
[Non-Patent Document 2] Grundy S M, Stone N J, Bailey A L, et al. 2018 AHA/ACC/AACVPR/AAPA/ABC/ACPM/ADA/AGS/APhA/ASPC/NLA/PCNA Guideline on the Management of Blood Cholesterol: A Report of the American College of Cardiology/American Heart Association Task Force on Clinical Practice Guidelines. Circulation. 2019 Jun. 18; 139(25): e1082-e1143
[Non-Patent Document 3] Authors/Task Force Members 2019 ESC/EAS guidelines for the management of dyslipidaemias: Lipid modification to reduce cardiovascular risk. Atherosclerosis 2019 November; 290: 140-205
[Non-Patent Document 4] Rodrigo Alonsol, Ada Cuevas1, Alberto Cafferata. Diagnosis and Management of Statin Intolerance. J Atheroscler Thromb, 2019; 26: 207-215
[Non-Patent Document 5] Statin Intolerance Clinical Guide Working Group: Kouji Kajinami, Kazuhisa Tsukamoto, Shinji Koba, et al., Statin Intolerance Clinical Guide 2018

### SUMMARY OF THE INVENTION

An object of the present invention is to provide a preventive and/or therapeutic agent for a disease on which a therapeutic effect can be expected by reduction in blood LDL-C as well as to provide a novel agent for reducing blood LDL-C.

To achieve the above-mentioned object, the present inventors made energetic investigations, and as a result, found that to their complete surprise, a compound disclosed as Example 85 in Patent Document 1 described above, that is, (R)-2-[3-[[N-(benzoxazol-2-yl)-N-3-(4-methoxyphenoxy)propyl]aminomethyl]phenoxy]butyric acid (hereinafter sometimes referred to as “compound A” or “pemafibrate”) reduced a blood LDL-C concentration, and was hence useful in the prevention and/or treatment of a disease on which a therapeutic effect was expected by reduction in blood LDL-C. In this way, the inventors completed the present invention.

That is, the present invention provides the following [1] to [28].

[1] An agent for reducing blood LDL cholesterol, comprising, as an active ingredient, (R)-2-[3-[[N-(benzoxazol-2-yl)-N-3-(4-methoxyphenoxy)propyl]aminomethyl]phenoxy]butyric acid or a salt thereof, or a solvate thereof.

[2] The agent for reducing blood LDL cholesterol according to [1], wherein the agent for reducing blood LDL cholesterol is intended for administration to a patient in whom a reduction in blood LDL cholesterol with a statin is insufficient or a patient in whom administration of the statin is restricted.

[3] An agent for preventing and/or treating hypercholesterolemia, comprising, as an active ingredient, (R)-2-[3-[[N-(benzoxazol-2-yl)-N-3-(4-methoxyphenoxy)propyl]aminomethyl]phenoxy]butyric acid or a salt thereof, or a solvate thereof.

[4] An agent for preventing and/or treating hyper-LDL-cholesterolemia, comprising, as an active ingredient, (R)-2-[3-[[N-(benzoxazol-2-yl)-N-3-(4-methoxyphenoxy)propyl]aminomethyl]phenoxy]butyric acid or a salt thereof, or a solvate thereof.

[5] The agent according to [3] or [4], which is intended for administration to a patient in whom a reduction in blood LDL cholesterol with a statin is insufficient or a patient in whom administration of the statin is restricted.

[6]A pharmaceutical composition for preventing and/or treating hypercholesterolemia, comprising a therapeutically effective dose of (R)-2-[3-[[N-(benzoxazol-2-yl)-N-3-(4-methoxyphenoxy)propyl]aminomethyl]phenoxy]butyric acid or a salt thereof, or a solvate thereof.

[7]A pharmaceutical composition for preventing and/or treating hyper-LDL-cholesterolemia, comprising a therapeutically effective dose of (R)-2-[3-[[N-(benzoxazol-2-yl)-N-3-(4-methoxyphenoxy)propyl]aminomethyl]phenoxy]butyric acid or a salt thereof, or a solvate thereof.

[8] The pharmaceutical composition according to [6] or [7], wherein the pharmaceutical composition is intended for administration to a patient in whom a reduction in blood LDL cholesterol with a statin is insufficient or a patient in whom administration of the statin is restricted.

[9]A method of reducing blood LDL cholesterol, comprising administering an effective dose of (R)-2-[3-[[N-(benzoxazol-2-yl)-N-3-(4-methoxyphenoxy)propyl]aminomethyl]phenoxy]butyric acid or a salt thereof, or a solvate thereof to a patient in need thereof.

[10] The method according to [9], wherein the patient is a patient in whom a reduction in blood LDL cholesterol with a statin is insufficient or a patient in whom administration of the statin is restricted.

[11] A method of preventing and/or treating hypercholesterolemia, comprising administering an effective dose of (R)-2-[3-[[N-(benzoxazol-2-yl)-N-3-(4-methoxyphenoxy)propyl]aminomethyl]phenoxy]butyric acid or a salt thereof, or a solvate thereof to a patient in need thereof.

[12]A method of preventing and/or treating hyper-LDL-cholesterolemia, comprising administering an effective dose of (R)-2-[3-[[N-(benzoxazol-2-yl)-N-3-(4-methoxyphenoxy)propyl]aminomethyl]phenoxy]butyric acid or a salt thereof, or a solvate thereof to a patient in need thereof.

[13] The method according to [11] or [12], wherein the patient is a patient in whom a reduction in blood LDL cholesterol with a statin is insufficient or a patient in whom administration of the statin is restricted.

[14] Use of (R)-2-[3-[[N-(benzoxazol-2-yl)-N-3-(4-methoxyphenoxy)propyl]aminomethyl]phenoxy]butyric acid or a salt thereof, or a solvate thereof for reducing blood LDL cholesterol.

[15] The use according to [14], wherein the (R)-2-[3-[[N-(benzoxazol-2-yl)-N-3-(4-methoxyphenoxy)propyl]aminomethyl]phenoxy]butyric acid or a salt thereof, or a solvate thereof is administered to a patient in whom a reduction in blood LDL cholesterol with a statin is insufficient or a patient in whom administration of the statin is restricted.

[16] Use of (R)-2-[3-[[N-(benzoxazol-2-yl)-N-3-(4-methoxyphenoxy)propyl]aminomethyl]phenoxy]butyric acid or a salt thereof, or a solvate thereof for preventing and/or treating hypercholesterolemia.

[17] Use of (R)-2-[3-[[N-(benzoxazol-2-yl)-N-3-(4-methoxyphenoxy)propyl]aminomethyl]phenoxy]butyric acid or a salt thereof, or a solvate thereof for preventing and/or treating hyper-LDL-cholesterolemia.

[18] The use according to [16] or [17], wherein the (R)-2-[3-[[N-(benzoxazol-2-yl)-N-3-(4-methoxyphenoxy)propyl]aminomethyl]phenoxy]butyric acid or a salt thereof, or a solvate thereof is administered to a patient in whom a reduction in blood LDL cholesterol with a statin is insufficient or a patient in whom administration of the statin is restricted.

[19] Use of (R)-2-[3-[[N-(benzoxazol-2-yl)-N-3-(4-methoxyphenoxy)propyl]aminomethyl]phenoxy]butyric acid or a salt thereof, or a solvate thereof for production of an agent for reducing blood LDL cholesterol.

[20] The use according to [19], wherein the agent for reducing blood LDL cholesterol is an agent for reducing blood LDL cholesterol for administration to a patient in whom a reduction in blood LDL cholesterol with a statin is insufficient or a patient in whom administration of the statin is restricted.

[21] Use of (R)-2-[3-[[N-(benzoxazol-2-yl)-N-3-(4-methoxyphenoxy)propyl]aminomethyl]phenoxy]butyric acid or a salt thereof, or a solvate thereof for production of a pharmaceutical composition for preventing and/or treating hypercholesterolemia.

[22] Use of (R)-2-[3-[[N-(benzoxazol-2-yl)-N-3-(4-methoxyphenoxy)propyl]aminomethyl]phenoxy]butyric acid or a salt thereof, or a solvate thereof for production of a pharmaceutical composition for preventing and/or treating hyper-LDL-cholesterolemia.

[23] The use according to [21] or [22], wherein the pharmaceutical composition is a pharmaceutical composition for administration to a patient in whom a reduction in blood LDL cholesterol with a statin is insufficient or a patient in whom administration of the statin is restricted.

[24] (R)-2-[3-[[N-(Benzoxazol-2-yl)-N-3-(4-methoxyphenoxy)propyl]aminomethyl]phenoxy]butyric acid or a salt thereof, or a solvate thereof for use in reduction of blood LDL cholesterol.

[25] The (R)-2-[3-[[N-(benzoxazol-2-yl)-N-3-(4-methoxyphenoxy)propyl]aminomethyl]phenoxy]butyric acid or a salt thereof, or a solvate thereof according to [24], wherein the (R)-2-[3-[[N-(benzoxazol-2-yl)-N-3-(4-methoxyphenoxy)propyl]aminomethyl]phenoxy]butyric acid or a salt thereof, or a solvate thereof is intended for administration to a patient in whom a reduction in blood LDL cholesterol with a statin is insufficient or a patient in whom administration of the statin is restricted.

[26] (R)-2-[3-[[N-(Benzoxazol-2-yl)-N-3-(4-methoxyphenoxy)propyl]aminomethyl]phenoxy]butyric acid or a salt thereof, or a solvate thereof for use in prevention and/or treatment of hypercholesterolemia.

[27] (R)-2-[3-[[N-(Benzoxazol-2-yl)-N-3-(4-methoxyphenoxy)propyl]aminomethyl]phenoxy]butyric acid or a salt thereof, or a solvate thereof for use in prevention and/or treatment of hyper-LDL-cholesterolemia.

[28] The (R)-2-[3-[[N-(benzoxazol-2-yl)-N-3-(4-methoxyphenoxy)propyl]aminomethyl]phenoxy]butyric acid or a salt thereof, or a solvate thereof according to [26] or [27], wherein the (R)-2-[3-[[N-(benzoxazol-2-yl)-N-3-(4-methoxyphenoxy)propyl]aminomethyl]phenoxy]butyric acid or a salt thereof, or a solvate thereof is intended for administration to a patient in whom a reduction in blood LDL cholesterol with a statin is insufficient or a patient in whom administration of the statin is restricted.

### Advantageous Effects of Invention

The present invention provides a novel drug useful in the prevention and/or treatment of a disease on which a therapeutic effect can be expected by a reduction in blood LDL-C. According to the present invention, it is possible to provide a novel agent for reducing blood LDL-C to a patient in whom a reduction in blood LDL-C with a statin is insufficient or a patient in whom the administration of the statin is restricted, and a novel preventive and/or therapeutic alternative to a patient of the disease on which the therapeutic effect can be expected by the reduction in blood LDL-C.

### BRIEF DESCRIPTION OF DRAWINGS

FIG. 1 shows the rate of a change in fasting blood LDL-C concentration at the time of the administration of the compound A (0.4 mg per day) to a patient of a nonalcoholic fatty liver disease.

FIG. 2 shows the rate of a change in fasting blood LDL-C concentration at the time of the administration of the compound A (0.4 mg per day) to the patient of the nonalcoholic fatty liver disease.

FIG. 3 shows the rates of changes from the baselines of a fasting blood lathosterol concentration, a fasting blood sitosterol concentration, and a fasting blood campesterol concentration at the time of the administration of the compound A (0.4 mg per day) to the patient of the nonalcoholic fatty liver disease.

### EMBODIMENTS FOR CARRYING OUT THE INVENTION

(R)-2-[3-[[N-(Benzoxazol-2-yl)-N-3-(4-methoxyphenoxy)propyl]aminomethyl]phenoxy]butyric acid (compound A) to be used in the present invention is represented by the following chemical formula (A):

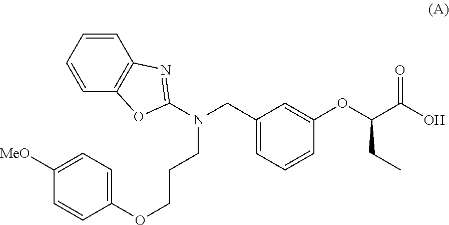 [SMILES: CC[C@@H](Oc1cccc(CN(CCCOc2ccc(OC)cc2)c2nc3ccccc3o2)c1)C(=O)O]

The compound A may be produced in accordance with, for example, a method described in WO 2005/023777 A1. The compound A may be formulated into a preparation in accordance with a method described in an academic literature. A preparation containing the compound A is approved as a therapeutic agent for hyperlipidemia “PARMODIA (registered trademark) TABLET” in Japan, and the “PARMODIA TABLET” may be used.

In one embodiment of the present invention, a salt or a solvate of the compound A may be used. The salt and the solvate may each be produced by an ordinary method. While the salt of the compound A is not particularly limited as long as the salt is pharmaceutically acceptable, examples thereof include: alkali metal salts, such as a sodium salt and a potassium salt; alkaline earth metal salts, such as a calcium salt and a magnesium salt; organic base salts, such as an ammonium salt and a trialkylamine salt; mineral acid salts, such as a hydrochloride and a sulfate; and organic acid salts such as an acetate. Examples of the solvate of the compound A or of the salt thereof include a hydrate and an alcohol adduct (e.g., an ethanol adduct).

As described in Examples described later, it has been shown that the compound A reduces a blood LDL-C concentration, and reduces both a synthesis marker and an absorption marker for cholesterol. It is conceivable from the foregoing that the compound A has the following effect: the compound suppresses the synthesis of LDL-C in a body and the absorption of cholesterol from a diet to reduce blood LDL-C. Accordingly, the compound A or a salt thereof, or a solvate thereof may serve as an active ingredient for an agent for reducing blood LDL-C. In addition, the compound A or a salt thereof, or a solvate thereof is useful in the prevention and/or treatment of a disease on which a therapeutic effect can be expected by a reduction in blood LDL-C through the reduction in blood LDL-C concentration.

Unless otherwise stated, the term “agent for reducing blood LDL-C” as used herein refers to, for example, a drug for a disease or the like on which a therapeutic effect can be expected by a reduction in blood LDL-C through its application. Specifically, the term refers to, for example, a drug for a disease or the like on which a therapeutic effect can be expected by a reduction in blood LDL-C through the inhibition of the synthesis of LDL-C in a living organism or the absorption of cholesterol therein. The mode of the reduction in LDL-C is not particularly limited, and examples thereof include: the suppression of the synthesis of the LDL-C; the inhibition of the absorption of the cholesterol; and the promotion of the excretion of the cholesterol.

The term “disease on which a therapeutic effect can be expected by a reduction in blood LDL-C” as used herein refers to, for example, hypercholesterolemia, familial hypercholesterolemia (homozygote), or familial hypercholesterolemia (heterozygote). Of those, hypercholesterolemia is preferred in the present invention, and hyper-LDL-cholesterolemia is more preferred.

The patients of those diseases include a patient on whom an existing agent for reducing LDL-C exhibits an insufficient effect such as a patient in whom a reduction in blood LDL-C with a statin is insufficient. In addition, the patients include a patient for whom treatment with the existing agent for reducing LDL-C is unsuitable such as a patient in whom the administration of the statin is restricted. Herein, the term “patient in whom a reduction in blood LDL-C with a statin is insufficient” refers to a patient in whom a reduction in blood LDL-C is not sufficiently achieved even under the administration of the statin. In addition, the term “patient in whom the administration of the statin is restricted” refers to a patient in whom the administration of the statin is restricted by contraindications, such as a muscle-related side effect and liver damage.

In the present invention, an administration subject is not particularly limited. However, from the viewpoint of providing a novel alternative of a drug therapy intended for a reduction in blood LDL-C, a patient in whom a reduction in blood LDL cholesterol with a statin is insufficient or a patient in whom the administration of the statin is restricted is preferred.

In the present invention, the compound A or a salt thereof, or a solvate thereof may be used alone because the compound A or a salt thereof, or a solvate thereof itself reduces a blood LDL-C concentration. However, in one aspect of the present invention, from the viewpoint of reducing blood LDL-C, the compound A or a salt thereof, or a solvate thereof may be used in combination with any other agent for reducing LDL-C intended for a reduction in blood LDL-C. Examples of such drug include an anion exchange resin, a statin, a small intestine cholesterol transporter inhibitor, and a PCSK9 inhibitor.

Examples of the anion exchange resin include colestyramine and colestimide. With regard to colestyramine, for example, QUESTRAN (registered trademark) powder 44.4% is available as a commercial drug. With regard to colestimide, for example, CHOLEBINE (registered trademark) tablet 500 mg is available as a commercial drug.

The statin is, for example, pravastatin, simvastatin, fluvastatin, atorvastatin, pitavastatin, rosuvastatin, or a salt thereof, or a solvate thereof.

The small intestine cholesterol transporter inhibitor is, for example, ezetimibe. With regard to ezetimibe, for example, ZETIA (registered trademark) tablet 10 mg is available as a commercial drug.

The PCSK9 inhibitor is, for example, a human anti-PCSK-9 monoclonal antibody preparation. With regard to the human anti-PCSK-9 monoclonal antibody preparation, for example, REPATHA (registered trademark) subcutaneous injection 140 mg pen is available as a commercial drug.

In one aspect of the present invention, the compound A or a salt thereof, or a solvate thereof, when used as a medicine, may be formulated into a preparation, such as a tablet, a capsule, a granule, a powder, a lotion, an ointment, an injection, or a suppository, through use of any other pharmaceutically acceptable carrier as required. Those preparations may each be produced by a known method. Examples of the pharmaceutically acceptable carrier may include an excipient, a disintegrator, a binder, a lubricant, a plasticizer, a glidant, a diluent, a solubilizer, a suspending agent, an isotonizing agent, a pH adjuster, a buffer, a stabilizer, a coating agent, a colorant, a taste-masking agent, and an odor-masking agent.

In one aspect of the present invention, the compound A or a salt thereof, or a solvate thereof may be administered by oral administration or parenteral administration, and oral administration is preferred. The therapeutically effective dose of the compound A or the salt thereof, or the solvate thereof, and the number of times of the administration thereof may be appropriately set by a person skilled in the art, though the dose and the number vary depending on, for example, the body weight, age, sex, and symptom of a patient. For example, for a normal adult, the compound A may be administered in a dose of from 0.05 mg to 0.8 mg per day in 1 to 3 portions, and is administered preferably in a dose of from 0.1 mg to 0.4 mg per day in 1 or 2 portions, more preferably in a dose of from 0.2 mg to 0.4 mg per day in 1 or 2 portions.

### EXAMPLES

The present invention is more specifically described below by way of Examples. However, these Examples are not intended to limit the present invention.

### Example 1: Investigation on Effect of Compound a on Blood LDL-C

An effect on blood LDL-C at the time of the oral administration of 0.4 mg per day of the compound A (a 0.2-milligram tablet of the compound A was administered twice a day at the time of fasting, 58 cases) to a patient of a nonalcoholic fatty liver disease (NAFLD) for 12 weeks was investigated by comparison to the administration of a placebo (60 cases).

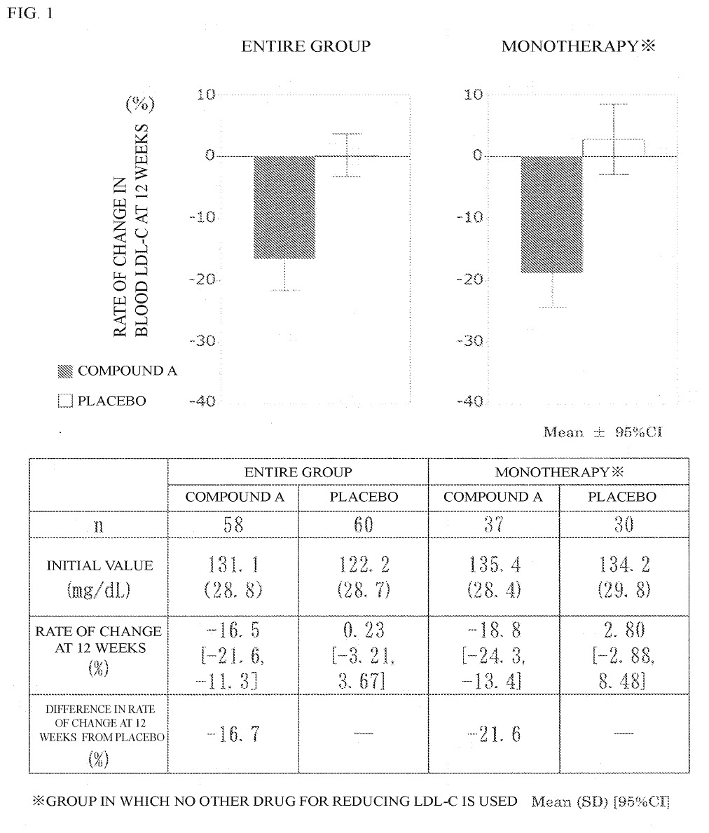

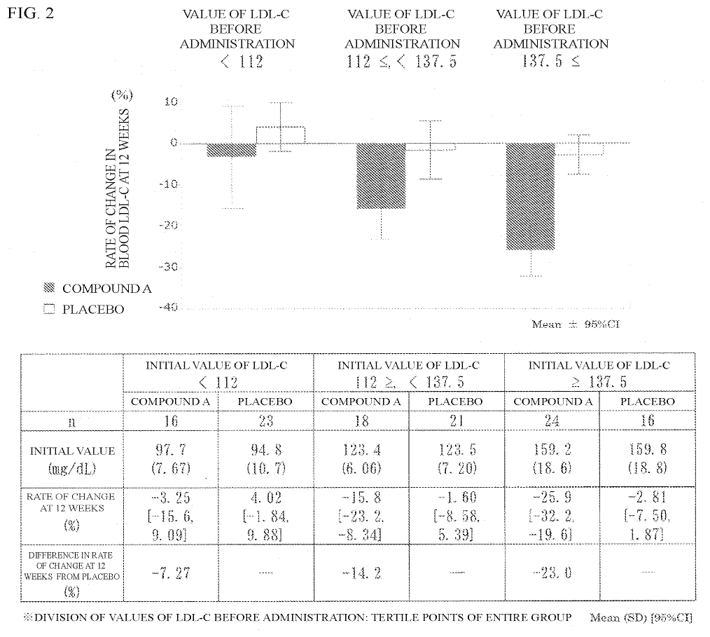

FIG. 1 shows the rate (%) of a change from the baseline of a blood LDL-C concentration at the time point of 12 weeks after the start of the administration. FIG. 2 shows the rate (%) of a change for each initial value of the blood LDL-C.

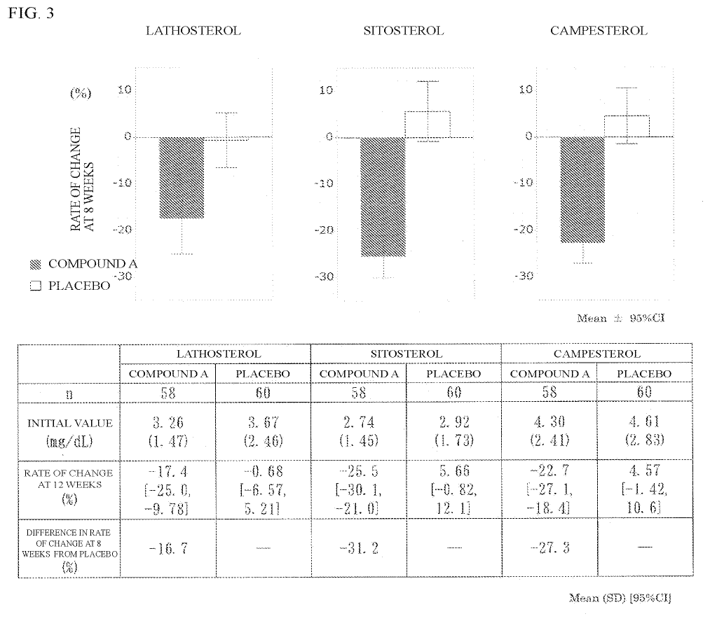

FIG. 3 shows the rate (%) of a change from the baseline of a blood lathosterol concentration, the rate (%) of a change from the baseline of a blood sitosterol concentration, and the rate (%) of a change from the baseline of a blood campesterol concentration at the time point of 8 weeks after the start of the administration.

It was made clear from FIG. 1 that in both an entire group including patients taking an existing agent for reducing LDL-C and patients who did not take any existing agent for reducing LDL-C, the administration of the compound A reduced a fasting blood LDL-C concentration as compared to the placebo administration. It was also made clear from FIG. 2 that the reducing effect of the compound A on LDL-C became larger as the blood LDL-C concentration before the administration thereof increased.

It was made clear from FIG. 3 that the administration of the compound A reduced the fasting blood concentration of lathosterol serving as a synthesis marker for cholesterol as compared to the placebo administration. It was also made clear that the administration of the compound A reduced the fasting blood concentration of sitosterol and the fasting blood concentration of campesterol, the sterols each serving as an absorption marker for cholesterol, as compared to the placebo administration.

### INDUSTRIAL APPLICABILITY

The present invention was completed on the basis of the fact that the inventors were the first to find the following, and the present invention is useful as a pharmaceutical drug for preventing and/or treating a disease on which a therapeutic effect can be expected by a reduction in blood LDL-C: the compound A has a clear reducing effect on a fasting blood LDL-C concentration.

1-5. (canceled)

6: A method of reducing blood LDL cholesterol, comprising administering an effective dose of (R)-2-[3-[[N-(benzoxazol-2-yl)-N-3-(4-methoxyphenoxy)propyl]aminomethyl]phenoxy]butyric acid or a salt thereof, or a solvate thereof to a patient subject in need thereof.

7: The method according to claim 6, wherein the subject is a patient in whom a reduction in blood LDL cholesterol with a statin is insufficient or a patient in whom administration of the statin is restricted.

8: A method of preventing and/or treating hypercholesterolemia, comprising administering an effective dose of (R)-2-[3-[[N-(benzoxazol-2-yl)-N-3-(4-methoxyphenoxy)propyl]aminomethyl]phenoxy]butyric acid or a salt thereof, or a solvate thereof to a subject in need thereof.

9: A method of preventing and/or treating hyper-LDL-cholesterolemia, comprising administering an effective dose of (R)-2-[3-[[N-(benzoxazol-2-yl)-N-3-(4-methoxyphenoxy)propyl]aminomethyl]phenoxy]butyric acid or a salt thereof, or a solvate thereof to a subject in need thereof.

10: The method according to claim 8, wherein the patient is a patient subject in whom a reduction in blood LDL cholesterol with a statin is insufficient or a patient in whom administration of the statin is restricted.

11-20. (canceled)

21: The method of claim 9, wherein the subject is a patient in whom a reduction in blood LDL cholesterol content when treated with a statin is insufficient or to whom the administration of a statin is restricted.
